(19) United States
(12) Patent Application Publication
(10) Pub. No.: US 2008/0085532 A1
(43) Pub. Date: Apr. 10, 2008

(54) Method for determining the immune status of a subject

(76) Inventors: Jorn Gorlach, Manchester, NJ (US); Jenifer Marie Gorlach, Manchester, NJ (US)

Correspondence Address: Jane Massey Licata;Licata & Tyrrell P.C., 66 E. Main Street, Marlton, NJ, 08053, US

(21) Appl. No.: 11/856,925

(22) Filed: Sep. 18, 2007

Related U.S. Application Data
(60) Provisional application No. 60/845,395, filed on Sep. 18, 2006.
(60) Provisional application No. 60/845,395, filed on Sep. 18, 2006.

(51) Int. Cl. C12Q 1/02; G01N 33/53

(52) U.S. Cl. 435/29; 436/501; 436/63

(57) ABSTRACT

The present invention is a method for using levels of soluble Clusters of Differentiation (CD) proteins, or cell surface-localized CD proteins extracted from T lymphocytes for determining the immune status of a subject. The present invention also a kit containing a CD protein extraction means and at least one antibody which specifically binds a CD protein for use in carrying out the method of the invention.

### INTRODUCTION

This application claims benefit of priority to U.S. Provisional Patent Application Ser. No. 60/845,395, filed Sep. 18, 2006, the content of which is incorporated herein by reference in its entirety.

### BACKGROUND OF THE INVENTION

Clinical evaluation of a patient infected with HIV virus (Acquired Immunodeficiency Syndrome; AIDS) currently relies on physician evaluation of patient status in conjunction with quantitation of T lymphocytes present in the patient's blood. Such assays have shown that HIV viral infection differentially affects the various blood cell types. Persons infected with the HIV virus, and particularly those persons characterized as having Acquired Immune Deficiency (AIDS) or Aids Related Complex (ARC) have been shown to have reduced numbers of CD4⁺ cells and, in many cases, elevated proportions of CD8⁺ cells. Thus, conventional approaches to diagnosing HIV infection have included monitoring CD4⁺ count, CD4⁺% and CD4⁺/CD8⁺ ratio.

However, cell counts require elaborate machinery such as Fluorescence Activated Cell Sorting (FACS) to determine the relative proportions of the various lymphocyte classes in a patient's blood. Such machines are costly and not readily available in developing countries. Moreover, these machines generally do not provide rapid and point-of-care analysis. Needed in the art is a low-cost or portable testing system and method for detecting and monitoring HIV infection. The present invention meets this need in the art.

### SUMMARY OF THE INVENTION

The present invention is a method for determining the immune status of a subject. The method involves extracting from a subject sample at least one soluble Clusters of Differentiation (CD) protein, or at least one cell surface-localized CD protein from T lymphocytes; and determining the amount of the CD protein, wherein said determined amount is indicative of the immune status of the subject. In particular embodiments, the CD protein is CD3, CD4, or CD8, or combinations thereof. In other embodiments, the method includes the prestep of selecting for T lymphocytes. A kit for determining the immune status of a subject is also provided.

### DETAILED DESCRIPTION OF THE INVENTION

The present invention relates to the use of cell surface proteins, as opposed to cells expressing such proteins, as markers for determining the immune status of a subject, i.e., determining whether the subject exhibits characteristics of immunosuppression, autoimmunity or hyperimmunity or other malfunction of the immune system. Subjects benefiting from such analysis include those infected with HIV or subjects with an autoimmune disease. In this regard, the instant method is distinct from the flow cytometry cell sorting-based methods conventional in the art. By using a protein-based assay, established immunological-based detection methods such as ELISA can be employed thereby reducing the cost and increasing the availability and speed of HIV diagnosis and AID progression to developing countries.

Given that specific Clusters of Differentiation (CD) molecules show a broad distribution curve if counted on individual cells, average CD cell surface protein amounts in a sample can be indicative of levels of T lymphocyte subsets. The instant method thus relates to CD cell surface protein amounts as they correlate with T lymphocyte counts and the immune status of a subject. It is contemplated that the direct quantification of the CD cell surface proteins in a sample is indicative of CD4⁺ count (i.e., total CD4⁺ cells per sample volume), CD4⁺% (i.e., total CD4⁺ cells per sample volume as percent of total CD3⁺ cells per sample volume) and CD4⁺/CD8⁺ ratio (i.e., total CD4⁺ cells per sample volume/total CD8⁺ cells per sample volume). Accordingly, particular embodiments embrace quantification of CD3, CD4, or CD8 protein amounts or combinations thereof.

In carrying out the method of the present invention, one or more soluble or T lymphocyte-localized cell surface CD proteins are extracted from a sample obtained from a subject, e.g., a subject having or suspected of having an HIV infection or other disease or condition in which immune status is indicative of disease or successful treatment thereof. In embodiments pertaining to the extraction of T lymphocyte-localized cell surface CD proteins, the sample contains white blood cells (e.g., whole blood). In embodiments pertaining to soluble CD proteins, the sample can be whole blood, plasma or serum. When employing whole blood, it may be desirable that the red blood cells be removed by hemolysis or, alternatively, that the white blood cells be selected from the other constituents of whole blood.

When extracting a soluble CD protein from a sample, it is desirable that an immobilized antibody which specifically binds the desired CD protein is employed. To illustrate, it is contemplated that soluble CD3 can be extracted from blood plasma by passing the plasma sample through a membrane or bead having an anti-CD3 antibody bound thereto. Antibody-coated surfaces such as beads or membranes are commercially available. See, e.g., DYNABEADS (DYNAL, Inc., Lake Success, N.Y.). Moreover, it is contemplated that peptide aptamers and molecular imprinting (see, e.g., U.S. Pat. No. 5,821,311) methods can be used to bind soluble CD proteins. In particular embodiments, quantification of shed CD3, CD4, and/or CD8 proteins provides a reliable determination of immune status.

Extraction of a cell surface-localized CD protein from a T lymphocyte is achieved using any suitable CD protein extraction means including both physical (e.g., sonication, osmolarity, etc.) and chemical means (e.g., immunoprecipitation, detergents, proteases, etc.). For example, CD4 extraction can be achieved using a buffer containing TRITON X-100 and Cytochalasin D which have been shown to solubilize CD4 from membranes (Foti, et al. (2002) Proc. Natl. Acad. Sci. USA 99:2008-2013). Further, immunoprecipitation is useful in the extraction of CD4 (Lynch, et al. (1996) Electrophoresis 17(1):227-34). CD3 gamma, delta and epsilon chain solubilization is known in the art (see Van Neerden, et al. (1990) Eur. J. Immunol. 20(9):2105-11). For example, NP-40 has been shown to completely remove CD3 from the cell surface (Geppert & Lipsky (1991) J. Immunol. 146(10):3298-305). Given the association of CD8 with the cytoskeleton (Geppert & Lipsky supra), it is contemplated that CD8 extraction can be achieved by combining a detergent such as TRITON X-100 or NP-40 with Cytochalasin D. In general, membrane-bound CD proteins of use in accordance with the instant invention can be extracted using combinations of sonication, change in pH, change in osmotic pressure, detergents, agents which disrupt the cytoskeleton, and immunoprecipitation with the appropriate antibody. Alternatively, the extracellular domain of the CD protein can be extracted from the T lymphocyte via protease cleavage. Accordingly, particular embodiments of the present invention embrace portions of the CD protein, in particular the extracellular portion (i.e., that portion of the protein located on the outside of the cell).

In particular embodiments of the method of the present invention, cell surface-localized CD proteins are extracted from T lymphocytes which have been selected or enriched for from the sample. Methods for selecting or enriching cells of interest are routinely practiced in the art. In general, such methods employ capturing cells via an antibody-coated bead or on an antibody-coated sample preparation device (e.g., tip of a syringe, membrane allowing flow through or flow by of other blood constituents and consequential elution). Such means for enrichment are commercially available and well-known to the skilled artisan. See, for example, EASYSEP Cell Enrichment Kits (Pierce, Rockford, Ill.) or DYNABEADS (DYNAL, Inc., Lake Success, N.Y.). In particular embodiments, CD3⁺, CD4⁺, and/or CD8⁺ cells are selected.

When enriching for multiple, individual cell types, a device which is coated with the different capturing antibodies can be employed. By way of illustration, three staggered rings at the end of a syringe can be each individually coated with a specific capturing antibody, and arranged in the order of selection, e.g., CD4, CD8, and CD3. Upon disassembly, each ring will have captured an individual cell type (i.e., CD4, CD8, or CD3) for subsequent extraction as described above.

Having extracted the CD protein(s) of interest, or portions thereof, from the sample, the amount of the CD protein(s) is determined using any suitable method for detecting and quantifying proteins. Desirably, protein quantitation is carried out by immunoassays using antibodies which specifically bind to the CD protein(s) of interest, or portion thereof. However, it is contemplated that artificial antibodies, peptide aptamers and molecular imprinting (see, e.g., U.S. Pat. No. 5,821,311) can be employed to detect and quantitate CD proteins in accordance with the present method.

Antibodies which specifically bind the CD proteins can be either polyclonal or monoclonal. Moreover, such antibodies can be natural or partially or wholly synthetically produced. All fragments or derivatives thereof which maintain the ability to specifically bind to the CD protein are also included. The antibodies can be a member of any immunoglobulin class, including any of the human classes: IgG, IgM, IgA, IgD, and IgE.

Antibody fragments can be any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, diabody, or Fd fragments. The antibody fragment can be produced by any means. For instance, the antibody fragment can be enzymatically or chemically produced by fragmentation of an intact antibody or it can be recombinantly produced from a gene encoding the partial antibody sequence. The antibody fragment can optionally be a single-chain antibody fragment. Alternatively, the fragment can be multiple chains which are linked together, for instance, by disulfide linkages. The fragment can also optionally be a multi-molecular complex. A functional antibody fragment typically contains at least about 50 amino acids and more typically contains at least about 200 amino acids.

An antibody for use in the method of the present invention can be generated using classical cloning and cell fusion techniques. For example, the antigen of interest is typically administered (e.g., intraperitoneal injection) to wild-type or inbred mice (e.g., BALB/c) or transgenic mice which produce desired antibodies, or rats, rabbits or other animal species which can produce native or human antibodies. The antigen can be administered alone, or mixed with adjuvant, or expressed from a vector (VEE replicon vector), or as DNA, or as a fusion protein to induce an immune response. Fusion proteins contain the peptide against which an immune response is desired coupled to carrier proteins, such as histidine tag (his), mouse IgG2a Fc domain, β-galactosidase, glutathione S-transferase, keyhole limpet hemocyanin (KLH), or bovine serum albumin, to name a few. In these cases, the peptides serve as haptens with the carrier proteins. After the animal is boosted, for example, two or more times, the spleen is removed and splenocytes are extracted and fused with myeloma cells using the well-known processes (Kohler and Milstein (1975) Nature 256:495-497; Harlow and Lane (1988) Antibodies: A Laboratory Manual, Cold Spring Harbor Laboratory, New York). The resulting hybrid cells are then cloned in the conventional manner, e.g., using limiting dilution, and the resulting clones, which produce the desired monoclonal antibodies, are cultured.

Alternatively, antibodies which specifically bind a CD protein are produced by a phage display method. Methods of producing phage display antibodies are well-known in the art (e.g., Huse, et al. (1989) Science 246(4935):1275-81).

Selection of CD-specific antibodies is based on binding affinity and can be determined by various well-known immunoassays including, enzyme-linked immunosorbent, immunodiffusion chemiluminescent, immunofluorescent, immunohistochemical, radioimmunoassay, agglutination, complement fixation, immunoelectrophoresis, and immunoprecipitation assays and the like which can be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904).

Antigens for antibody production can be produced using any conventional eukaryotic or prokaryotic expression system. Such systems are well-known and routinely employed in the art. Moreover, commercial sources such as INVITROGEN, CLONTECH, STRATAGENE and PROMEGA provide a variety of different vectors and host cells for producing recombinant proteins, without and with tags (e.g., FLAG, His6, etc.) to facilitate purification. Nucleic acid molecules which encode human CD3, CD4, and CD8 proteins are well-known in the art. For example, GENBANK Accession Nos. NM_{—}000616 and X provide the nucleic and amino acid sequence for CD4 and CD8, respectively. The CD3 antigen is composed of 5 polypeptide chains gamma (GENBANK Accession No. NM_{—}000073), delta (GENBANK Accession No. NM_{—}000732), epsilon (GENBANK Accession No. NM_{—}012099), zeta (GENBANK Accession No. NM_{—}198053) and eta. As such, an antibody can be produced to any one of these chains. Indeed antibodies have been successfully produced to the epsilon chain set forth in GENBANK Accession No. NM_{—}012099.

In determining the amount of a particular CD protein, the extracted protein is contacted with an antibody which specifically binds the CD protein. The antibody is allowed to bind to the CD protein to form an antibody-antigen complex. The conditions and time required to form the antibody-antigen complex may vary and are dependent on the sample being tested and the method of detection being used. Once non-specific interactions are removed by, for example, washing the sample, the antibody-antigen complex is detected using any one of the immunoassays described above as well a number of well-known immunoassays used to detect and/or quantitate antigens (see, for example, Harlow and Lane (1988) supra). Such well-known immunoassays include antibody capture assays, antigen capture assays, and two-antibody sandwich assays. In particular embodiments, when an antibody is used to immunoprecipitate the CD protein from the sample, a second antibody which specifically binds the CD protein is employed in a sandwich assay to detect and quantitate the CD protein.

Immunoassays of the present invention typically rely on labeled antibodies, or secondary reagents for detection. These antibodies can be labeled with radioactive compounds, enzymes, biotin, or fluorochromes. Of these, radioactive labeling can be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and can be labeled with radioisotopes or enzymes. Fluorochromes, although requiring expensive equipment for their use, provide a very sensitive method of detection. Those of ordinary skill in the art will know of other suitable labels which can be employed in accordance with the present invention. The binding of these labels to antibodies or fragments thereof can be accomplished using standard techniques (e.g., Kennedy, et al. (1976) Clin. Chim. Acta 70:1-31; Schurs, et al. (1977) Clin. Chim. Acta 81:1-40) and methods of detecting these labels are also well-known to the skilled artisan.

After detecting the level of CD protein present in a sample, the results seen in a given subject can be compared with a known standard or the absolute levels can be used for determining immune status. A known standard can be a statistically significant reference group of normal subjects or subjects that have an HIV infection to provide diagnostic, prognostic, or predictive information pertaining the subject from whom the sample was obtained. The standard can be generated by performing analyses of multiple samples derived from multiple stages of HIV progression. A known standard can also be a reference sample taken form the same subject at another time. Based upon this comparison to the standard, the progression of the HIV infection can be determined.

When employing absolute levels of CD protein (e.g., CD4, CD3 and/or CD8), it is desirable that ethnic or population baselines be established for each CD protein.

It is contemplated that levels of membrane-bound CD proteins and/or shed (i.e., soluble) portions of the corresponding CD proteins can be used as a reliable measurement for HIV disease progression. For example, low CD4 protein levels in a test subject, compared to CD4 protein levels in a healthy individual, would be indicative of an impaired immune system and therefore an increase risk of being susceptible to, e.g., an opportunistic infection. Moreover, it is contemplated that any marker or collection of markers, independent of the previously disclosed CD3, CD4, and CD8 proteins, can be used in the disclosed method as long as the marker has a reproducible quantitative relationship to the cell count of the associated T-cells or the progression of HIV infection.

Given that the specific Clusters of Differentiation show a broad distribution curve if counted on individual cells and that the instant method will fulfill requirements concerning reliability, reproducibility, and medical validity of the results (e.g., standard deviation of measurements), large numbers of individual cells can be analyzed. However, because the instant method involves the measurement of individual marker proteins instead of cell counts, sample volumes necessary for statistical relevance can be reduced since most cells carry more than one, and even more than 1000, individual markers per cell.

By way of illustration, when one considers conventional cell count analysis of a typical sample volume (e.g., ˜100 μl), 40,000 CD4⁺ cells are analyzed based on a total of 60,000 CD3⁺ T lymphocytes. By measuring the individual CD proteins as set forth in the instant method, the number of detectable entities analyzed increases by the factor 10-100; therefore as few as 600 or 6000 CD3⁺ T lymphocytes are required in accordance with the present method. If CD3⁺ cells are selected (e.g., through hemolysis and filtration or bead extraction) the number of detectable entities could be increased even further. Given this statistic background, a variation of the distribution of specific CD proteins on individual cells can be overcome, and the average marker count should have a strong, reliable relationship to the absolute cell count.

From a translation perspective, a linear relationship would be the most straightforward. However, a non-linear relationship of average marker concentration to average cell count could even increase the ability to determine differentiation in immune status. If, e.g., the average concentration of a CD protein per cell decreases with the decrease of the cell type characterized by that CD protein, and the depletion of that cell type is a marker for disease progression, then a measurement of the specific CD protein instead of a cell count would give a stronger “signal” to determine HIV disease progression. The same is true for a cell type marker that exhibits an increase in average concentration, wherein the cell type increases in number during disease progression.

The present invention also provides a kit which is useful for carrying out the method of the present invention. The kit generally contains a container containing kit a CD protein extraction means and at least one antibody which specifically binds a CD protein. CD protein extraction means includes physical extraction (e.g., sonication, high temperature, and changes in osmolarity) or detergents (e.g., TRITON X-100 or NP-40) used alone or in combination with each other or agents which facilitate cellular and/or cytoskeleton disruption (e.g., Cytochalasin D). In particular embodiments, the kit contains anti-CD proteins antibodies such as anti-CD3, anti-CD4, or anti-CD8, or combinations thereof. In some embodiments, the kit contains a capture antibody and a detection antibody for each individual CD proteins. The kit can also contain other solutions necessary or convenient for carrying out the invention, e.g., chelating agents, protease inhibitors, membranes, pipette tips, and the like. The container can be made of glass, plastic or foil and can be a vial, bottle, pouch, tube, bag, etc. The kit can also contain written information, such as procedures for carrying out the present invention; analytical information, such as the amount of reagent contained in the first container; or data describing the correlation between the level of CD protein in a sample and the progression of HIV infection. The container can further be in another container, e.g., a box or a bag, along with the written information.

It is contemplated that the instant method of measuring the amount of membrane-bound CD proteins for determining the immune status of a subject can be applied to CD proteins still bound to the membrane (i.e., the CD proteins have not been extracted from the cell). For example, antibody-bound beads or membranes can be used to capture cells expressing on their surface the CD protein of interest, and a detection antibody can be employed to determine the amount of CD protein present.

It is further contemplated that the instant method can be adapted to other markers of CD4⁺, CD3⁺, and/or CD8⁺ cells, wherein the amount of said markers can are representative, respectively, of the CD4⁺, CD3⁺, and/or CD8⁺ cell counts.

What is claimed is:

1. A method for determining the immune status of a subject comprising extracting from a subject sample at least one soluble Clusters of Differentiation (CD) protein, or at least one cell surface-localized CD protein from T lymphocytes; and determining the amount of the CD protein, wherein said determined amount is indicative of the immune status of the subject.

2. The method of claim 1, wherein the CD protein is CD3, CD4, or CD8, or combinations thereof.

3. The method of claim 1, further comprising the prestep of selecting for T lymphocytes.

4. A kit for determining the immune status of a subject comprising a CD protein extraction means and at least one antibody which specifically binds a CD protein.

5. The kit of claim 4, wherein the CD protein is CD3, CD4, or CD8, or combinations thereof.
